(19) United States
(12) Patent Application Publication
(10) Pub. No.: US 2024/0033220 A1
(43) Pub. Date: Feb. 1, 2024

(54) METHOD AND DEVICE FOR PRODUCING A LIQUID CONTAINING LIPOSOMES, AND PRODUCED LIQUID

(71) Applicant: Fraunhofer-Gesellschaft zur Förderung der angewandten Forschung e.V., Munich (DE)

(72) Inventors: Regina Bleul, Mainz (DE); Raphael Thiermann, Mainz (DE)

(73) Assignee: Fraunhofer-Gesellschaft zur Förderung der angewandten Forschung e.V., Munich (DE)

(21) Appl. No.: 18/320,487

(22) Filed: May 19, 2023

Related U.S. Application Data
(63) Continuation of application No. PCT/EP2021/082320, filed on Nov. 19, 2021.

(30) Foreign Application Priority Data
Nov. 19, 2020 (DE) 102020214601.2

(51) Int. Cl. A61K 9/127; A61K 47/24

(52) U.S. Cl. CPC A61K 9/1277; A61K 47/24

(57) ABSTRACT

A method and a device for producing a liquid containing liposomes is presented. The method comprises the conduction of a first liquid and a second liquid into a micromixer and to the output of the micromixer by gas pressure from at least one source of gas. The total flow rate of the liquids is adjusted in such a way that the total flow rate at the output of the micromixer is at least 10 mL/min. This allows the production of liquids, which contain liposomes having a narrow size distribution, on an industrial scale in a simple and reproducible manner. The embodiments also include a liquid containing liposomes having a narrow size distribution and to uses thereof.

### PRIORITY

This application is a Continuation of PCT/EP2021/082320, filed on Nov. 19, 2021, entitled “METHOD AND DEVICE FOR PRODUCING A LIQUID CONTAINING LIPOSOMES, AND PRODUCED LIQUID”, published as WO 2022/106627 A1, which claims priority to German Application No. DE 10 2020 214 601.2, filed Nov. 19, 2020, the entireties of both are incorporated herein by reference.

### BACKGROUND

Liposomes are the most established nanotransporter systems for pharmaceutical applications worldwide. Doxil® was already approved by the FDA as the first “nanodrug” in 1995.

Currently, nano-liposomes are produced on an industrial scale in a multi-step batch method. In a first step, the lipids, mostly large, multilamellar liposomes (MLV), are hydrated. In a second step “downsizing” is performed to obtain nanoliposomes with a diameter of <200 nm. For this purpose, extrusion is carried out an high pressure through a membrane, which has corresponding nanosized pores (Example: production of Doxil®). Alternatively, high-pressure homogenization is performed (Examples: production of AmbiSome® and nanoliposomes used in cosmetics).

The extrusion needs to carried out at high temperatures, as the MLV lipid bilayers need to be flexible enough to allow for changes in shape which are necessary to achieve a reduction in size. Multiple passages through the extrusion membrane are necessary to achieve the required narrow size distribution. This procedure is time-consuming. In addition, this procedure is limited to heat-resistant lipid raw materials and corresponding incorporated or encapsulated substances. In addition, the extrusion method involves a loss of material at the membrane. Furthermore, a clogging (blocking) of the membranes requires frequent replacement of the membranes and loss of potentially valuable substances, such as lipids and active ingredients, which compromises and increases the cost of implementing a production method for providing a sterile liquid with liposomes. The commonly used polycarbonate membranes also have batch fluctuations due to different properties such as pore size, pore uniformity and surface wetting, which results in poor reproducibility of the process.

High-pressure homogenization on the other hand often results in distributions that are too broad in size, including the production of large percentages of very small liposomes. In addition, the high-pressure homogenization also has to be carried out at high temperatures.

There is a need for a method for the rapid and simple, large-scale production of a sterile liquid which contains liposomes in the nanometer size range. Furthermore, there is a need for formulation methods for thermolabile (sensitive) lipids and drugs. There is also a need for a platform technology for next-generation drugs, such as e.g. nucleic acid-based immunotherapeutics.

WO 2017/103268 A1 discloses a continuous method for producing nanoparticles. The method disclosed there is not suitable for the large-scale production of sterile liquids containing liposomes.

EP 1337322 B1 discloses a method for producing lipid vesicles. This method is not a strictly microfluidic method and is limited with respect to the use of sophisticated APIs (e.g. ultrahydrophobic agents) in the production of liposomes. Furthermore, there is still room for improvement regarding the achieved narrowness of the size distribution of the produced liposomes.

WO 2014/172045 A1 discloses a method for the industrial production of sterile liposome solutions on a large scale. However, the reproducibility is problematic, because the production is carried out via a platform, in which an absolutely even distribution of the different channels of the platform has to be ensured, which is not easy.

### SUMMARY

A method and a device are provided for producing a liquid containing liposomes. The method comprises directing a first liquid and a second liquid into a micromixer and up to the outlet of the micromixer by gas pressure from at least one gas source, optionally in addition by at least one device for delivering liquid, wherein the total flow rate of the liquids is adjusted such that it is at least 10 mL/min at the outlet of the micromixer. The method and the device allow liquids to be provided in a simple and reproducible manner on an industrial scale, which contain liposomes with a narrow size distribution. Furthermore, a liquid containing liposomes with a narrow size distribution is provided and uses of the latter are proposed.

### BRIEF DESCRIPTION OF THE DRAWINGS

The device according to the embodiments will be described in more detail with reference to Figures, which illustrate examples of those embodiments. The same or different reference numerals may be used for the same or similar elements in the Figures and their explanation may be omitted in part. Non-limiting and non-exhaustive embodiments are described with reference to the following drawings. The components in the drawings are not necessarily to scale, emphasis instead being placed upon illustrating the principles of the invention. The drawings, like referenced numerals, designate corresponding parts throughout the different views.

FIG. 1 shows a device according to one embodiment including a single micromixer 3.

FIG. 2 shows a device according to one embodiment including a first micromixer 3 and a second micromixer 12.

FIG. 3 shows a device according to one embodiment when the delivery of the liquids is not only performed by gas pressure from sources of gas, but is also supported by a device for the delivery of liquids.

FIG. 4 shows a device for transporting liquids including a magnetic gear pump and a 3-way valve is arranged downstream of the outlet of the second micromixer.

### DETAILED DESCRIPTION

The object of the present invention was to provide a method and a device for producing a liquid containing liposomes, which do not have the disadvantages of the prior art. In particular, the method and the device should make it possible to provide liquids on an industrial scale in a simple and reproducible manner, which contain liposomes with a narrow size distribution and which are in particular sterile.

The object is solved by the embodiments described herein. According to the embodiments, a continuous method is provided for producing a liquid containing liposomes, comprising the steps: providing a first liquid in a first container, wherein the first liquid comprises at least one lipid or consists of the latter; providing a second liquid in a second container, wherein the second liquid comprises water or consists of the latter; directing the first liquid along a first fluid line into a first inlet of a micromixer and in a flow up to an outlet of the micromixer; directing the second liquid along a second fluid line into a second inlet of the micromixer and in a flow adjacent to the first liquid up to the outlet of the micromixer; wherein the first liquid and the second liquid mix inside the micromixer, so that a liquid containing liposomes is discharged at the outlet of the micromixer; wherein directing the first liquid and the second liquid into the micromixer and up to the outlet of the micromixer is performed by gas pressure from at least one gas source, optionally in addition by at least one device for delivering liquid (e.g. a magnetically driven pump, preferably selected from the group consisting of gear pump, gear ring pump or centrifugal pump), wherein the total flow rate of the liquids is adjusted so that it is at least 10 mL/min at the outlet of the micromixer.

According to the embodiments, the term “liposomes” is defined as liposomes, lipoplexes and lipid nanoparticles, which can optionally be charged with a substance (e.g. an active ingredient), wherein “charged” is defined to mean that a cavity within the lipid particles and/or a membrane of the lipid particles (preferably both) comprise or contain a substance (e.g. an active ingredient). The liposomes (i.e. the liposomes, lipoplexes and/or lipid nanoparticles) have in particular a diameter in the range of 20 nm to <200 nm or in the range of >200 nm to <500 nm, preferably in the range of 40 nm to 150 nm or in the range of 250 nm to 400 nm, particularly preferably in the range of 60 nm to 120 nm or in the range of 300 nm to 350 nm. The diameter can be determined by dynamic light scattering and/or cryogenic transmission electron microscopy, preferably by a measurement with cryogenic transmission electron microscopy.

The term “micromixer” is preferably defined as all mixers, whose mixing principle is based on the mixing principle of a micromixer, including those which are so large (scaled) that their fluid channels have larger dimensions (i.e. cross-sections) than the micrometer range (range of 1 μm to 1000 μm).

By the method according to the embodiments, it is possible to provide liquids containing liposomes with narrow size distribution in a simple and reproducible manner on an industrial scale. The method is also suitable for ensuring that sterile liquids containing liposomes can be provided, as the liquid delivery is performed by gas pressure from at least one gas source (optionally in addition by at least one device for liquid delivery). The optional liquid delivery device is arranged downstream of the at least one gas source and it can be ensured that its surfaces in contact with the liquid are sterile. By outwardly sealing the components used in the method (e.g. the fluid lines and the micromixer), it can also be ensured that the liquids do not come into contact with microorganisms and/or viruses.

The mixing of the liquids in a micromixer allows a high degree of control over the structuring of the liposomes, i.e. it is possible to obtain excellent size control and very narrow side distributions can be achieved. A further advantage is that the method can be scaled up without having to be readjusted, i.e. without for example having to produce an even distribution of the flows during the “numbering-up” of the individual micromixers, which is often necessary for micromixers (“external numbering-up”) and/or without the mixer type having to be changed. When scaling up the method, for example a larger mixer of the same type (“scalable micromixer”) can be used (e.g. a Caterpillar 600 instead of a Caterpillar 300, StarLam 300 instead of a StarLam 30), i.e. the effort involved in changing the procedure or system configuration is thus significantly lower or eliminated altogether. In the case of the StarLam micromixer, this increase is also referred to as an “internal numbering-up”. This is a critical advantage especially for GMP methods. Scalable micromixers, which can be used according to the embodiments as mixers or micromixers, are characterized in particular in that, in the case of scaling, they do not require any additional distribution lines and manifolds. Scalable micromixers are for example the ramp-up/ramp-down split and recombine mixers, here in particular the aforementioned caterpillar-type micromixers (e.g. as disclosed in Hermann et al., Chemical Engineering Journal, vol. 334, p. 1996-2003), StarLam-type micromixers (e.g. as disclosed in DE 199 27 556 C2) and/or cyclone mixers (e.g. as disclosed in EP 1 390 131 B1).

The method may include that all surfaces with which the first and the second liquid come into contact on their way to the outlet of the micromixer are sterile. Furthermore, it is preferred that all of these surfaces are outwardly fluid-tight, preferably forming a closed system. The advantage here is that for the micromixer and the fluid delivery devices (e.g. the gas pressure from at least one gas source, optionally the additional device for liquid delivery) the conditions can be set very exactly, which is advantageous for achieving desired PDI values and other quality criteria. In addition, it can be ensured that the used liquids do not come into contact with microorganisms and/or viruses. Apart from this, it is preferred that all of these surfaces do not have any regions where residues can collect. It is also preferred that all of these surfaces do not comprise glass or consist of glass. Each of these features helps to ensure that the method can be used to continuously produce a sterile liquid containing liposomes, i.e. no contamination of the liquid can occur during its production. In a preferred embodiment, all fluids used in the method are sterile (e.g. the first and second sterile liquid and the gas from the gas source). The same can apply for all components used in the method (e.g. micromixers, conveyors and containers), at least for their surfaces which come into contact with the fluids used in the method.

The method may include the at least one gas source comprises or consists of a gas container. The at least one gas source can have a first fluidic connection to the first container and a second fluidic connection to the second container. In addition, the at least one gas source can contain a gas which does not include oxygen, wherein the gas preferably comprises or consists of a gas selected from the group consisting of nitrogen, noble gas and mixtures thereof.

The gas pressure of the gas source provides a delivery pressure, optionally together with a device for liquid delivery. The total flow rate can be adjusted here via a constant gas pressure of the gas source, optionally also via the device for liquid delivery. The gas pressure alone is preferably in the region of <12 bar, more preferably <8 bar, particularly preferably <6 bar, most preferably greater than 1 to 6 bar, in particular 1.5 to 5 bar. With the optional device for liquid delivery, delivery pressures of up to 50 bar are achieved. The total flow rate can then be kept constant via at least one, preferably at least one first and at least one second, flow regulator, wherein particularly preferably the at least one first flow regulator is arranged at the first fluidic connection and the at least one second flow regulator is arranged at the second fluidic connection. In brief, the at least one flow regulator is used to convert the initial delivery pressure into a constant flow rate. In flow direction, first the gas source (then optionally the device for liquid delivery) and then the at least one flow regulator are arranged.

The loss of pressure in the mixing chamber of the mixer is preferably low, preferably in the range of <6 bar, in particular in the range of 1.5 to 5 bar.

In addition, the total flow rate can be adjusted so that, at the outlet of the micromixer, it is ≥80 mL/min, preferably ≥320 mL/min, particularly preferably ≥1280 mL/min, most preferably ≥2800 mL/min, in particular ≥5120 mL/min.

Apart from this, the total flow rate can be adjusted so that, at the outlet of the micromixer per cross-sectional area of the micromixer, it is ≥20 ml/(min-mm2), preferably ≥100 ml/(min-mm2), particularly preferably ≥200 ml/(min-mm2), most preferably ≥400 ml/(min-mm2), optionally ≥1000 ml/(min-mm2), in particular 100 to 400 ml/(min-mm2).

Furthermore, the total flow rate can be adjusted so that the ratio of the flow rate of the second liquid to the flow rate of the first liquid is <100:1, preferably <20:1, particularly preferably <16:1, most preferably <8:1, even more preferably <7:1, strongly preferably <6:1, very strongly preferably <5:1, in particular ≤4:1.

The total flow rate can have a flow rate variation of less than 1% of the total flow rate, preferably less than 0.1% of the total flow rate. The advantage here is that very low PDI values are achieved. Furthermore, the total flow rate can be adjusted such that the flow has a Reynolds number in the range of >80 to <1200, preferably >120 to <1000.

The method may include that the micromixer has one or more mixing structures extending obliquely or transversely to the flow direction, which are preferably suitable for deflecting the first liquid and/or second liquid in a direction obliquely or transversely to the flow direction.

Furthermore, the micromixer, particularly preferably all of the components used in the method, can comprise or consist of stainless steel. The advantage here is that the micromixer or all components used in the method can easily be sterilized by the effect of temperature and a cleaning validation can be established. Consequently, the micromixer does not need to be a one-off product, for which the reproducible mixing performance has to be controlled and checked constantly. The same also applies to other components used in the method, i.e. for the whole device for performing the method, if the latter comprises or consists of stainless steel.

In addition the micromixer can be autoclaved. Apart from this, the micromixer can be taken apart into at least two parts for cleaning fluid channels of the micromixer.

The micromixer can be a “split and recombine” micromixer, wherein the micromixer is preferably a “ramp-up/ramp-down” micromixer, particularly preferably a “caterpillar”-type micromixer (see e.g. Hermann et al., Chemical Engineering Journal, vol. 334, p. 1996-2003). It has been found that “caterpillar”-type micromixers have multiple advantages. Firstly, they have a continuous channel. This is in contrast to many other “split and recombine” micromixers, in which the main channel splits for example into fluidically separated channels, which then join together again. In addition, the production and cleaning of the “caterpillar”-type micromixers is simpler. In addition, the shearing forces that occur in these micromixers are lower, as the repeated changes in direction along the oblique surfaces in flow direction are only quite gentle. Preferably, the oblique surfaces are inclined relative to the main flow direction (i.e. with respect to a flow direction parallel to the walls of the fluid channels of the micromixer) by less than 70°, particularly preferably less than 55° and most preferably less than 45°. Consequently, the production of the liquid containing the liposomes is gentler, i.e. there is less degradation of the educts and the products. Furthermore, this micromixer can be scaled very easily, for example by increasing the cross-sectional area of the mixing channel perpendicular to the main flow direction while maintaining the repeating basic structure typical of the caterpillar-type micromixer. With increasing enlargement, the mixing performance can be adjusted, if necessary by increasing the number of repeating basic structures.

The micromixer can also be a “split in micro-lamellae and combine in multilaminated stream” micromixer, particularly preferably a “StarLam” micromixer (see e.g. DE 199 27 556 C2 and Werner, B. et al., Chemical Engineering Technology, 2005, vol. 28, p. 401 ff). This has the advantage that it can also be made of stainless steel, is easy to assemble and disassemble and is also easily scalable.

Preferably, the micromixer, in particular the “caterpillar” micromixer, downstream of the mixing chamber, has a substantially non-constricting and/or substantially straight outlet. The advantage here is that there is no abrupt change in direction and/or cross-sectional narrowing of the fluid flow, no dead spots and only a small loss of pressure and low shearing forces.

The method may include that the first liquid comprises lipids in a total concentration of >30 g/L, preferably >50 g/L, particularly preferably >80 g/L, most preferably >150 g/L, in particular 160 g/L to 400 g/L (optionally 210 g/L to 290 g/L).

Furthermore, the first liquid can comprise at least one phospholipid, preferably at least one zwitterionic or anionic phospholipid, wherein the phospholipid is preferably selected from the group consisting of phosphatidylcholine, DSPC, DOPE, DOPC, DSPE, HSPC and mixtures thereof, wherein the concentration of the at least one phospholipid, or mixtures thereof, is preferably >20 g/L, preferably >40 g/L, particularly preferably >80 g/L, most preferably >160 g/L, in particular 210 g/L to 400 g/L. Furthermore, the first liquid can comprise phospholipids with saturated fatty acid residues and/or unsaturated fatty acid residues, such as DSPC (saturated) and DOPC (unsaturated), or mixtures thereof.

In addition, the first liquid can comprise at least one PEGylated lipid, preferably DSPE-PEG2000 and/or DMG-PEG2000, wherein the concentration of the PEGylated lipid is preferably in the range of 15 to 40 Mol. %, particularly preferably in the range of 31 to 35 Mol. %, with respect to the molar amount of at least one phospholipid in the first liquid.

In addition, the first liquid can comprise at least one lipid, preferably at least one cationic lipid (e.g. a pH-dependent cationically charged lipid). In particular, substances selected from the group consisting of DOTMA (1,2-di-O-octadecenyl-3-trimethylammonium propane), DOTAP (1,2-dioleoyloxy-3-(trimethylammonium)propane), DDAB dimethyldioctadecylammonium (bromide salt), DODMA (1,2-dioleyloxy-3-dimethylaminopropane) (cationically charged at low pH) and mixtures thereof, wherein the concentration of the at least one lipid, or mixtures thereof, is preferably >10 g/L, preferably >20 g/L, particularly preferably >40 g/L, most preferably >80 g/L, in particular 90 g/L to 350 g/L.

In addition, the first liquid can comprise at least one lipidoid, wherein the concentration of the lipidoid is preferably in the range of 200 to 1000 Mol. %, particularly preferably in the range of 300 to 800 Mol. %, with respect to the molar amount of at least one lipid in the first liquid. The term “lipidoid” is defined according to the embodiments as lipid-like substances which result from the reaction of acrylamides or acrylic acid esters with secondary or primary amines (i.e. are produced). The pH-dependent cationic charge is preferably achieved by means of a primary, secondary or tertiary amino group. In particular, at least one lipidoid is meant which is selected from the group of lipidoids mentioned in the publication by Acinc, A. et al., Nature Biotechnology (2008), vol. 26, no. 5, p. 561-569.

In addition, the first liquid can comprise cholesterol. The first liquid may optionally not comprise a non-ionic, cationic, anionic and/or amphoteric surfactant, preferably no surfactant (at all). Furthermore, the first liquid can comprise at least one organic solvent or no organic solvent. If it comprises an organic solvent, the organic solvent is preferably an organic, water-miscible solvent, particularly preferably a solvent, which is selected from the group consisting of alcohols, particularly preferably ethanol, 1-propanol, 2-propanol, and/or methanol, acetone, tetrahydrofuran, dioxane, acetonitrile, dimethyl sulfoxide, in particular ethanol. In one embodiment, the first liquid is degassed. This prevents the formation of bubbles when assembling the liposomes.

The method may include that the second liquid comprises a buffer substance, preferably a buffer substance selected from the group consisting of acetate, ammonium, citrate and combinations thereof, particularly preferably calcium acetate and/or ammonium sulphate. The concentration of the buffer substance is preferably in the range of 5 mM to 300 mM, particularly preferably in the range of 8 mM to 250 mM. The advantage of these concentrations is that the formation of a gradient is improved.

In one embodiment, the second liquid is degassed. This prevents the formation of bubbles when assembling the liposomes. The method may include that the liposomes of the liquid are charged with at least one active substance, wherein the charging is preferably performed in the first micromixer, takes place in a further mixer downstream of the first micromixer, takes place after assembling the liposomes and/or takes place after purifying the liposomes.

Here, the active substance can comprise or consist of at least one organic active substance, preferably an active ingredient for treating a disease, particularly preferably a molecule selected from the group consisting of vitamin, protein, peptide, lipid, DNA, RNA, organic molecule with a mass ≤500 Da and mixtures thereof, in particular a substance selected from the group consisting of ultrahydrophobic substance, siRNA and combinations thereof. In this way, it is possible to encapsulate challenging active substances (e.g. ultrahydrophobic substances and/or siRNA), which could not be encapsulated in other methods. Furthermore, the active substance can comprise or consist of at least one inorganic active substance, preferably a substance selected from the group consisting of magnetic substance, paramagnetic substance and mixtures thereof, particularly preferably iron oxide, manganese oxide and mixtures thereof.

The at least one active substance is preferably contained in the first liquid, in the second liquid and/or in a further liquid (downstream of the micromixer). The concentration of the at least one organic active substance and/or at least one inorganic active substance can be ≥1 wt. %, preferably 5 to 80 wt. %, particularly preferably 10 to 60 wt. %, in particular 15 to 30 wt. %, with respect to the total weight of the lipids (or the components of the liposomes excluding water).

In step a), b) and/or c), preferably in steps a) to c), of the method, it can be tempered to a temperature of >10° C. to <70° C., preferably 15 to 40° C., particularly preferably 22 to 30° C., in particular 23° C. to 25° C. If necessary, it can be tempered to a temperature within a range of >0° C. to <10° C. This variant of the method has the advantage that temperature-sensitive substances (e.g. active ingredients) can be used in the method, i.e. liposomes can be provided which comprise these temperature-sensitive substances.

The method may include that the liposomes of the liquid, which are discharged at the outlet of the micromixer, are unilamellar liposomes, multilamellar liposomes or mixtures thereof.

The method can be configured so that the liposomes of the liquid which are discharged at the outlet of the micromixer have a diameter in the range of 20 nm to <200 nm or in the range of >200 nm to <500 nm, preferably in the range of 40 nm to 150 nm or in the range of 250 nm to 400 nm, particularly preferably in the range of 60 nm to 120 nm or in the range of 300 nm to 350 nm. The diameter can be determined by dynamic light scattering and/or cryogenic transmission electron microscopy, preferably by a measurement with cryogenic transmission electron microscopy.

Furthermore, the method may include that the liposomes of the liquid, which are discharged at the outlet of the micromixer, are more than 50%, more than 70%, or more than 90%, with respect to the total number of all liposomes in the liquid, unilamellar liposomes, and the liposomes, with respect to their size distribution, have a PDI value in the range of <0.200, preferably ≤0.150, particularly preferably in the region of ≤0.100, most preferably in the region of ≤0.090, in particular in the region of ≤0.075.

Alternatively, the method may include that the liposomes of the liquid, which are discharged at the outlet of the micromixer, are more than 50%, more than 70% or more than 90% multilamellar liposomes, with respect the to the total number of all liposomes in the liquid, and the liposomes, with respect to their size distribution, have a PDI value in the region of <0.500, preferably in the region of <0.300.

The PDI can preferably be determined or is determined by means of dynamic light scattering according to the standard DIN ISO 22412:2018-09.

The method may include that the liquid is purified in a further step after step c), the purification comprising, following step c), performing a, preferably continuous, ultrafiltration, gel filtration and/or evaporation, particularly preferably ultrafiltration, in order to enrich the liposomes. Furthermore, the purification can comprise removing substances which are present in addition to the liposomes, preferably removing buffer substances and/or organic solvents. In addition, the purification can comprises an exchange of substances which are present in addition to the liposomes for a sterile, osmolar sugar solution, preferably for a sterile, osmolar glucose solution or sucrose solution. The sterile, osmolar sugar solution particular preferably comprises 4 to 15 wt. % sugar, in particular 4 to 6 wt. % glucose in case of a glucose solution and/or 9 to 11 wt. % sucrose in case of a sucrose solution. The purification can comprise a sterilization of the solution containing liposomes, preferably comprising a sterile filtration through a membrane with a pore diameter (cut-off) of 0.2 μm.

The method can further comprise the following steps: directing the liquid containing liposomes at the outlet of the micromixer along a third fluid line, optionally a dwelling loop, into a first inlet of a further mixer and in a flow up to an outlet of the further mixer; and directing a further liquid along a fourth fluid line into a second inlet of the further mixer and in a flow adjacent to the liquid containing liposomes up to the outlet of the further mixer.

Hereby, the liquid containing liposomes is mixed with the further liquid inside the further mixer, so that a changed liquid containing liposomes is discharged at the outlet of the further mixer. Preferably, in this further mixer, the amount of solvent is reduced and a further dilution is performed, preferably a dilution to less than half, preferably less than a quarter, of the original concentration of liposomes in the liquid. In this way the liposomes therein are more dimensionally stable. Alternatively, also other parameters, such as for example the pH value, can be adjusted by the admixture, wherein preferably the pH value is reduced or neutralized. Furthermore, the liposomes can also be charged in this step with at least one active substance. Directing the liquid containing liposomes and the further liquid into the further mixer can be performed by gas pressure from at least one gas source, optionally also by at least one device for delivering liquid, wherein the total flow rate of the liquids is adjusted to be more than 10 mL/min, preferably at least 20 mL/min at the outlet of the further mixer. Preferably, the further mixer is a micromixer, preferably a static micromixer, particularly preferably a “split and recombine” micromixer, most preferably a “caterpillar”-type micromixer. Alternatively, also a so-called “StarLam” micromixer can be used. Both mixers can be easily adapted to the required flow rate by suitable selection or scaling.

In principle, the liposomes of the liquid can be charged with at least one active substance in the first micromixer, in the further mixer, after assembling the liposomes and/or after purifying the liposomes.

According to the embodiments, a device is provided for producing a liquid containing liposomes, comprising: a first container, which contains a first liquid, wherein the first liquid comprises or consists of at least one lipid; a second container, which contains a second liquid, wherein the second liquid comprises or consists of water; a micromixer, which has a first inlet, a second inlet and an outlet, wherein the first container is connected via a first fluid line to the first inlet of the micromixer and the second container is connected via a second fluid line to the second inlet of the micromixer, wherein the micromixer is configured to let the first liquid and the second liquid flow respectively in a flow inside the micromixer to the outlet of the micromixer and mix inside the micromixer, wherein a liquid containing liposomes is discharged at the outlet of the micromixer; at least one gas source, optionally also at least one device for delivering liquid; and a control unit; characterized in that the at least one gas source is configured to deliver the first liquid and the second liquid into the micromixer by gas pressure from the at least one gas source, optionally also by the at least one device for liquid delivery, wherein the control unit is configured, to adjust the total flow rate of the liquids so that it is at least 10 mL/min at the outlet of the micromixer.

The device may include that all surfaces of the device, with which the first and second liquids can come into contact on their way to the outlet of the micromixer, are sterile. Furthermore, all of these surfaces of the device can be outwardly fluid-tight, preferably forming a closed system. The advantage here is that for the micromixer and the fluid delivery devices (e.g. the gas pressure from at least one gas source, optionally the additional device for liquid delivery) the conditions can be set very exactly, which is advantageous for achieving desired PDI values and other quality criteria. In addition, it can be ensured that the used liquids do not come into contact with microorganisms and/or viruses. Apart from this, all these surfaces can be considered to have no regions where residues can collect. In addition, it is preferred that all of these surfaces do not comprise glass or consist of glass. Each of these features helps to ensure that the device can be used to continuously produce a sterile liquid containing liposomes, i.e. no contamination of the liquid can take place during its production. In a preferred embodiment all fluids present in the device (e.g. the first and second sterile liquid and the gas from the gas source) are sterile. The same can apply to all components of the device (e.g. micromixers, conveyors and containers), at least to their surfaces which come into contact with the fluids contained in the device.

The device may include that the device and/or the control unit of the device is/are configured to perform the method according to the embodiments. Here, the device can have features which are mentioned above in connection with the method according to the embodiments and/or the control unit of the device can be configured to perform steps which are mentioned above in a connection with the method according to the embodiments.

According to the embodiments, a liquid containing liposomes is provided which is characterized in that more than 50%, more than 70% or more than 90%, of all liposomes of the liquid are unilamellar liposomes, and the liposomes have, with regard to their size distribution, a PDI value in the region of <0.200, preferably in the region of ≤0.150, particularly preferably in the region of ≤0.100, most preferably in the region of ≤0.090, in particular in the region of ≤0.075; or more than 50%, more than 70% or more than 90%, of all liposomes of the liquid are multilamellar liposomes, and the liposomes have, with respect to their size distribution, a PDI value in the region of <0.500, preferably in the region of <0.300; wherein the PDI is preferably determined by means of dynamic light scattering according to the standard DIN ISO 22412:2018-09.

The smaller the PDI of the liposomes in the liquid, the higher the uniformity of the liposomes in terms of their size and other properties. For example, in an application of the liposomes (into a living body) a more uniform size of the liposomes makes their biodistribution more predictable and able to be controlled more precisely. In addition, a liquid which contains liposomes with a small PDI can better ensure that the liquid does not contain a dangerous amount of liposomes that are too large which are considered to be a risk factor for developing embolisms. A smaller PDI can thus also provide a higher degree of safety. In addition, the smaller the PDI the more uniform the stability of the liposomes, which allows more accurate statements to be made on the storability, transport stability and handling of the liposomes. Furthermore, a smaller PDI of the liposomes means that in the case of charging the liposomes with active ingredient, the active ingredient content per liposome is more uniform, which enables more precise dosing of the active ingredient.

The liquid can be produced by the method according to the embodiments. In a preferred embodiment, the provided liquid containing liposomes is sterile. The liquid is proposed for use in medicine, preferably for use in a method for the therapeutic treatment of the human or animal body, particularly for the application of an active ingredient, particularly preferably for the topical application of an active ingredient; and/or for targeting an active ingredient; and/or for releasing an active ingredient; in a method for the surgical or therapeutic treatment of the human or animal body, wherein the active ingredient is preferably selected from the group consisting of vaccine, cytostatic, corticoid and combinations thereof (doxorubicin and/or dexamethasone) and for treatment, in particular for the treatment of cancer, of an inflammatory disease, a disease of the immune system and/or a neurodegenerative disease. The active ingredient can optionally be at least one of the organic active substances mentioned above (in connection with the method according to the embodiments).

Furthermore, the liquid is proposed for use in a diagnostic method, preferably a diagnostic method which is performed on the human or animal body, in particular a diagnostic method in which the liposomes comprise a biomarker. In addition, the use of the liquid is proposed in cosmetics and/or as an additive in food products. In addition, the use of the liquid is proposed for encapsulating at least one substance, optionally in a first micromixer, in a further mixer, after assembling the liposomes and/or after purifying the liposomes; and/or for targeting at least one substance; and/or for releasing at least one substance; and/or producing a composite material; wherein the at least one substance preferably comprises or consists of at least one organic active substance and/or at least one inorganic active substance, and the use is optionally an in vitro use. The at least one organic active substance and/or the at least one inorganic active substance can be one of the active substances mentioned above (in connection with the method according to the embodiments).

With reference to the following Figures and Examples, the subject-matter according to the embodiments is explained in more detail, without being restricted to the specific embodiments shown here.

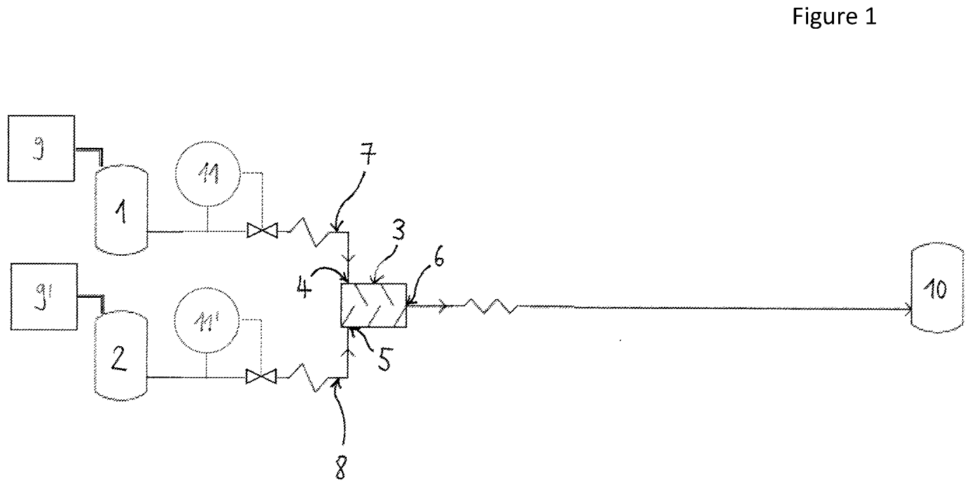

FIG. 1 shows a device according to the embodiments which comprises a single micromixer 3. The first liquid, which is located in a first container 1, and the second liquid, which is located in a second container 2, are introduced by gas pressure, which originates from two gas sources 9, 9′, via a first fluid line 7 into the first inlet 4 or via a second fluid line 8 into a second inlet 5 of the micromixer 3 and transported up to the outlet 6 of the micromixer. In the first fluid line 7 and in the second fluid line 8 a flow regulator 11, 11′ ensures that the liquid flow in these fluid lines 7, 8 is kept at a desired level. The outlet 6 of the first micromixer is fluidically connected to a reservoir 10, so that the liquid containing liposomes discharged from the first micromixer 3 is directed into the reservoir 10.

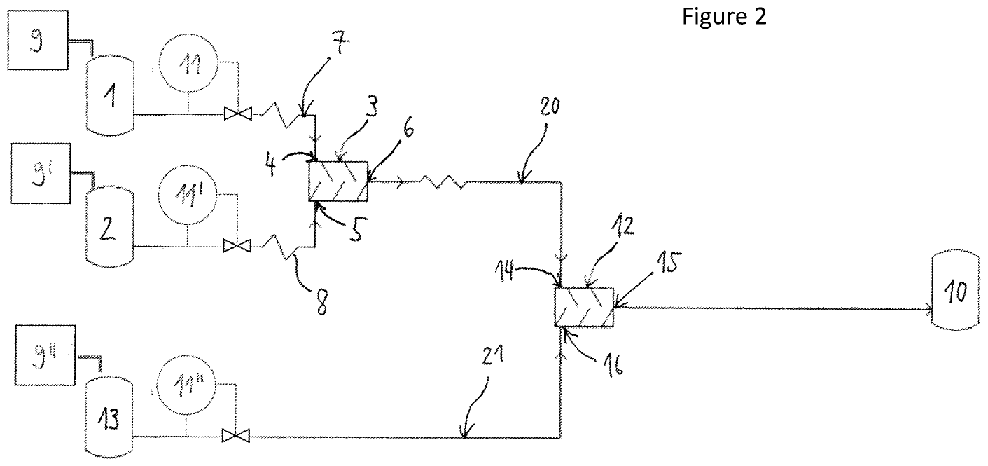

FIG. 2 shows a device according to the embodiments which comprises a first micromixer 3 and a second micromixer 12. The first liquid, which is located in a first container 1, and the second liquid, which is located in a second container 2, are introduced by gas pressure, which originates from two gas sources 9, 9′, via a first fluid line 7 into the first inlet 4 or via a second fluid line 8 into a second inlet 5 of the micromixer 3 and transported up to the outlet 6 of the micromixer. At the outlet of the micromixer 3 a liquid containing liposomes is discharged and is directed via a third fluid line 20 into a first inlet 14 of a further mixer 12 (here: a further micromixer). A further liquid, which is located in a third container 13, is directed by gas pressure, which originates from a gas source 9″, via a fourth fluid line 21 into a second inlet 16 of the further mixer 12. In the further mixer 12 the liquid containing liposomes is mixed with the further liquid, wherein the mixture is discharged at the outlet 15 of the second micromixer 12 and is directed into a reservoir 10. In the first fluid line 7, in the second fluid line 8 and in the fourth fluid line 21 a respective flow regulator 11, 11′, 11″ ensures that the liquid flow in these fluid lines 7, 8, 21 is kept at a desired level.

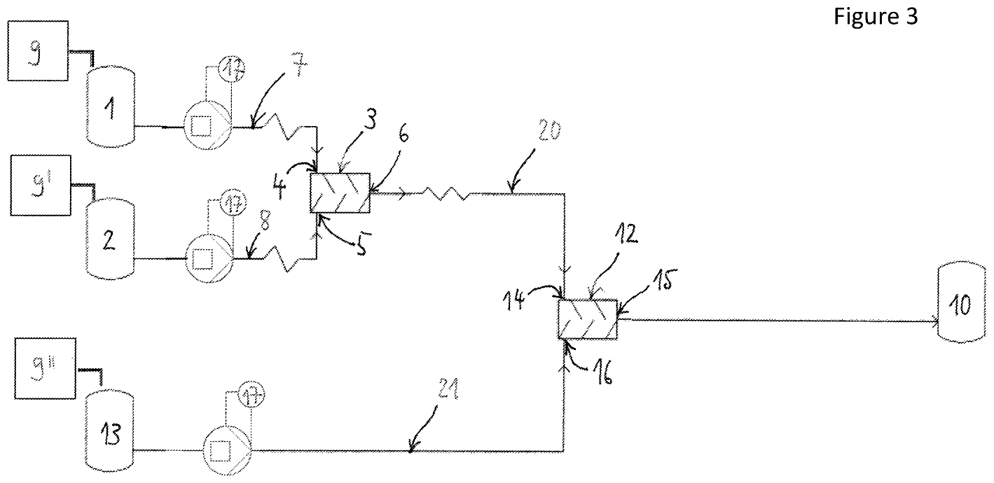

FIG. 3 shows a device according to the embodiments which is constructed like the device shown in FIG. 2, with the difference that the delivery of the liquids is not only performed by gas pressure from sources of gas, but is also supported by a device 17 for the delivery of liquids (here: a pump), in order to achieve the necessary delivery pressure. The device 17 for delivering liquids also has the property of a flow regulator and ensures that the respective flow of liquids in the first fluid line 7, the second fluid line 8 and the fourth fluid line 21 is kept at a desired level.

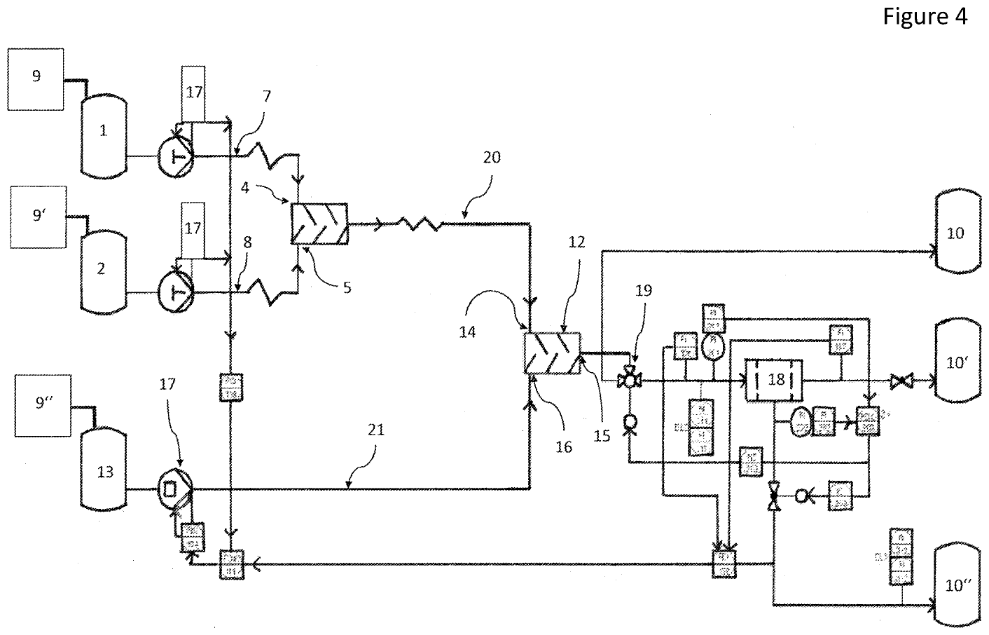

FIG. 4 shows a device according to the embodiments, which is essentially constructed like the device shown in FIG. 3, wherein the device 17 for transporting liquids is a magnetic gear pump and a 3-way valve 19 is arranged downstream of the outlet 15 of the second micromixer 12. Via the 3-way valve 19, the liquid containing liposomes discharged from the second micromixer 12 can either be directed into a reservoir 10 or via an ultrafiltration module 18 into another reservoir 10′, 10″.

### Example 1—Structure of a Device According to the Embodiments

The device comprises a micromixer and a second micromixer, which is connected behind a short dwelling loop at the outlet of the first micromixer, for example in order to achieve asymmetrical flow conditions, or to obtain dilution prior to purification by diafiltration to further reduce solvent content.

The temperature of the micromixer(s) and dwelling loop can be adjusted if necessary. A diafiltration module (membrane stack) can also be connected which can be operated directly in series or as a separate module in a circuit.

The micromixer is preferably a “split and recombine” micromixer, particularly preferably a “caterpillar” micromixer (continuous mixing channel with a plurality of mixing stages). The mixing channel of these micromixers has a diameter in the micrometer range to the millimeter range. Of course, “upscaling” is possible here. For example, the same product quality can be achieved using a Caterpillar 600 with 4-times the flow rate, and with a Caterpillar 1200 with 16-times the flow rate compared with R-300.

Examples of scalability:

Caterpillar 300:2-80 ml/min=22.22 ml/(min*mm²)−888.88 ml/(min*mm²)

Caterpillar 600:8-320 ml/min=22.22 ml/(min*mm²)−888.88 ml/(min*mm²)

Caterpillar 1200:32-1280 ml/min=22.22 ml/(min*mm²)−888.88 ml/(min*mm²)

Caterpillar 2400:128-5120 ml/min=22.22 ml/(min*mm²)−888.88 ml/(min*mm²)

Possible scaling to channel structure width (rounded) is:

20 ml/(min*mm²)−1000 ml/(min*mm²)
Scalable from 300-2400 Caterpillar: 0.12-345.6 L/h

Alternatively, the micromixer and/or the second micromixer can also be a so-called StarLam micromixer. The StarLam micromixer is also scalable and can be operated for example as a StarLam 30, 300 and 3000 with flow rates of 12 l/h to 8000 l/h.

### Example 2—Production of a Liquid Containing Liposomes

First liquid:
100 g/L (HSPC:PEG-lipid:cholesterol 3:1:1) in ethanol

Second liquid:
250 mM calcium acetate

The two liquids are mixed at ambient temperature with a R300 caterpillar. Advantage: This mixer operates virtually optimally in these flow conditions: Sufficient mixing, low drop in pressure, straight outlet, solid processing, low risk of blockage, comparatively easy to clean, opens in two halves for cleaning and drying.

Filling then takes place without purification. The liquid is stored in the refrigerator overnight.

The next day, the liquid is purified by ultrafiltration and the calcium acetate is replaced externally by 5% glucose solution (due to slightly lower viscosity compared to sucrose solution with the same osmolarity).

Properties of the liposomes in the liquid:
diameter (according to DLS): 80 nm
PDI: 0.011

### Example 3—Production of a Liquid Containing Liposomes which Encapsulate siRNA

The lipid mixture contained the lipidoid dodecyl-3-[3-[3-[3-[3-[bis(3-dodecoxy-3-oxo-propyl)amino]propyl-methyl-amino]propyl-(3-dodecoxy-3-oxo-propyl)amino]propanoate (M=1106.8 g/mol), 1,2-distearoyl-sn-glycero-3-phosphocholine (DSPC), cholesterol and 1,2-dimyristoyl-rac-glycero-3-methoxypolyethylene glycol-2000, (DMG-PEG 2000).

To produce a lipid solution the cationic lipidoid was combined with cholesterol, DSPC and DMG-PEG2000 and dissolved in 90% ethanol (10% 10 mM citrate buffer, pH 3) in the molar ratio of the cationic lipid, wherein the molar ratios of DSPC were: cholesterol:DMG-PEG2000 50:10:38.5:1.5.

To produce a polynucleotide solution an si-RNA polynucleotide (30 μM) was dissolved in 10 mM citrate buffer, pH 3.0.

To produce the lipoplex lipid nanoparticles the polynucleotide solution and the lipid solution were mixed in a first micromixer (total flow rate 10 ml/min).

The mixed product was mixed with PBS buffer in a second micromixer (total flow rate 20 ml/min).

The relative volumetric flow rates of polynucleotide solution: Lipid solution:Buffer was 1:1:2.

The freshly prepared lipid nanoparticles were dialyzed against PBS buffer, in order to remove ethanol, exchange buffer and unbound siRNAs.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The above disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description. While various embodiments of the invention have been described, it will be apparent to those of ordinary skill in the art that many more embodiments and implementations are possible within the scope of the invention. Accordingly, the invention is not to be restricted except in light of the attached claims and their equivalents.

### LIST OF REFERENCE SIGNS

1: first container;
2: second container;
3: micromixer;
4: first inlet of micromixer;
5: second inlet of micromixer;
6: outlet of micromixer;
7: first fluid line;
8: second fluid line;
9, 9′, 9″: gas source;
10, 10′, 10″: reservoir;
11, 11′, 11″: flow regulator;
12: further micromixer;
13: third container;
14: first inlet of further micromixer;
15: outlet of further micromixer
16: second inlet of further micromixer;
17: device for liquid delivery (e.g. magnetic gear pump);
18: ultrafiltration module;
19: 3-way valve;
20: third fluid line;
21: fourth fluid line.

1. A continuous method for producing a liquid with liposomes, comprising the steps:
a) providing a first liquid in a first container, wherein the first liquid comprises at least one lipid;
b) providing a second liquid in a second container, wherein the second liquid comprises water;
c) directing the first liquid along a first fluid line into a first inlet of a micromixer and in a flow up to an outlet of the micromixer; and
d) directing the second liquid along a second fluid line into a second inlet of the micromixer and in a flow adjacent to the first liquid up to the outlet of the micromixer;
wherein the first liquid and the second liquid mix inside the micromixer, so that a liquid with liposomes is discharged at the outlet of the micromixer; further wherein the directing of the first liquid and of the second liquid into the micromixer and up to the outlet of the micromixer is performed by gas pressure from at least one gas source, further wherein the total flow rate of the liquids is adjusted so that it is at least 10 mL/min at the outlet of the micromixer.

2. The method according to claim 1, wherein all surfaces with which the first and second liquid come into contact on their way to the outlet of the micromixer:
i) are sterile;
ii) are outwardly fluid-tight;
iii) have no regions where residues can collect; or
iv) do not comprise glass.

3. The method according to claim 1, wherein the at least one gas source comprises a gas container that comprises:
a first fluidic connection to the first container;
a second fluidic connection to the second container; and
a gas which does not contain oxygen, wherein the gas comprises one of: nitrogen, noble gas, or mixtures thereof.

4. The method according to claim 1, wherein the total flow rate:
i) is adjusted via a constant gas pressure of the gas source, also via a device for increasing pressure, which is in the region of <12 bar;
ii) is kept constant by at least one flow regulator;
iii) is adjusted so that, at the outlet of the micromixer, it is ≥80 mL/min;
iv) is adjusted so that, at the outlet of the micromixer, per cross-sectional area of the outlet of the micromixer, it is ≥20 ml/(min-mm²);
v) is adjusted so that the ratio of the flow rate of the second liquid to the flow rate of the first liquid is <8:1;
vi) has a flow rate variation of less than 1% of the total flow rate; or
vii) is configured such that the stream has a Reynolds number in the range of >80 to <1200.

5. The method according to claim 1, wherein the micromixer:
i) comprises one or more mixing structures extending obliquely or transversely to the flow direction, which are suitable for deflecting the first liquid and/or second liquid in a direction obliquely or transversely to the flow direction;
ii) comprises stainless steel or consists of stainless steel;
iii) can be autoclaved;
iv) can be separated into at least two parts for cleaning fluid channels of the micromixer; or
v) is a “split and recombine” micromixer or a “StarLam” micromixer.

6. The method according to claim 1, wherein the first liquid:
i) comprises lipids in a total concentration of >30 g/L;
ii) comprises at least one phospholipid;
iii) comprises at least one PEGylated lipid;
iv) comprises at least one lipid;
v) comprises at least one lipidoid;
vi) comprises cholesterol;
vii) does not contain a non-ionic, cationic, anionic and/or amphoteric surfactant;
viii) comprises at least one organic solvent or no organic solvent; or
ix) is degassed.

7. The method according to claim 1, wherein the second liquid:
i) comprises a buffer substance; or
ii) is degassed.

8. The method according to claim 1, wherein the liposomes of the liquid are charged with at least one active substance, wherein the at least one active substance comprises:
i) at least one organic active substance, including an active ingredient for treating a disease; or
ii) comprises at least one inorganic active substance.

9. The method according to claim 1, wherein in step a), b) and/or c), it is tempered to a temperature of >10° C. to <70° C.

10. The method according to claim 1, wherein the liposomes of the liquid exiting at the outlet of the micromixer:
i) are unilamellar liposomes, multilamellar liposomes or mixtures thereof;
ii) have a diameter in the range of 20 nm to <200 nm or in the range of >200 nm to <500 nm, wherein the diameter can be determined by dynamic light scattering or cryogenic transmission electron microscopy; or
iii) are to more than 50%, with respect to the total number of all liposomes in the liquid, unilamellar liposomes, and the liposomes, with respect to their size distribution, have a PDI value in the region of <0.200, or more than 50% with reference to the total number of all liposomes in the liquid, are multilamellar liposomes and the liposomes, with respect to their size distribution, have a PDI value in the range of <0.500, wherein the PDI can be determined or is determined by dynamic light scattering according to the standard DIN ISO 22412:2018-09.

11. The method according to claim 1, wherein the liquid is purified after c), wherein the purification comprises at least one of the following:
i) performing an ultrafiltration, gel filtration and/or evaporation to enrich the liposomes;
ii) removing substances, which are present in addition to the liposomes; and
iii) exchanging substances, which are present in addition to the liposomes, for a sterile, osmolar sugar solution.

12. The method according to claim 1, wherein the method further comprises:
i) directing the liquid with liposomes at the outlet of the micromixer along a third fluid line, optionally a dwelling loop, into a first inlet of a further mixer and in a flow up to an outlet of the further mixer; and
ii) directing a further liquid from a third container, along a fourth fluid line into a second inlet of the further mixer and in a flow adjacent to the liquid with liposomes up to the outlet of the further mixer;
wherein the liquid with liposomes and the further liquid mix inside the further mixer, so that at the outlet of the further mixer a changed liquid with liposomes is discharged;
wherein directing the liquid with liposomes and the further liquid into the further mixer is performed via gas pressure from at least one gas source, and/or via at least one device for delivering liquid, wherein the total flow rate of the liquids is adjusted so that at the outlet of the further mixer it is more than 10 mL/min.

13. A device for producing a liquid with liposomes, comprising:
a first container with a first liquid that comprises at least one lipid;
a second container with a second liquid that comprises water;
a micromixer comprising a first inlet, a second inlet and an outlet, wherein the first container is connected via a first fluid line to the first inlet of the micromixer and the second container is connected via a second fluid line to the second inlet of the micromixer, further wherein the micromixer is configured to allow the first liquid and the second liquid to flow respectively in a flow inside the micromixer up to the outlet of the micromixer and to mix inside the micromixer, wherein the liquid with the liposomes is discharged at the outlet of the micromixer;
at least one gas source; and
a control unit configured to cause the first liquid and the second liquid to flow into the micromixer and up to the outlet of the micromixer via gas pressure from the at least one gas source, wherein the total flow rate of the liquids is adjusted so that at the outlet of the micromixer it is at least 10 mL/min.

14. The device according to claim 13, wherein all surfaces of the device, with which the first and second liquids can come into contact on their way to the outlet of the micromixer:
are sterile;
are outwardly fluid-tight;
have no regions where residues can collect; or
do not comprise glass.

15. The device according to claim 13, wherein the device or the control unit are configured for:
providing a first liquid in a first container, wherein the first liquid comprises at least one lipid;
providing a second liquid in a second container, wherein the second liquid comprises water;
directing the first liquid along a first fluid line into a first inlet of a micromixer and in a flow up to an outlet of the micromixer; and
directing the second liquid along a second fluid line into a second inlet of the micromixer and in a flow adjacent to the first liquid up to the outlet of the micromixer;
wherein the first liquid and the second liquid mix inside the micromixer, so that a liquid with liposomes is discharged at the outlet of the micromixer; further wherein the directing of the first liquid and of the second liquid into the micromixer and up to the outlet of the micromixer is performed by gas pressure from at least one gas source, further wherein the total flow rate of the liquids is adjusted so that it is at least 10 mL/min at the outlet of the micromixer.

16. A liquid with liposomes, wherein:
more than 50% of all liposomes in the liquid are unilamellar liposomes and the liposomes have, with regard to their size distribution, a PDI value in the region of <0.200; or
more than 50% of all liposomes in the liquid are multilamellar liposomes and the liposomes have, with respect to their size distribution, a PDI value in the region of <0.500;
wherein the PDI is determined by dynamic light scattering according to the standard DIN ISO 22412:2018-09.

17. The liquid according to claim 15, wherein the liquid is used for therapeutic treatment of a human or animal body.

18. The liquid according to claim 15, wherein the liquid is used for a diagnostic method performed on the human or animal body.

19. The liquid according to claim 15, wherein the liquid is produced from a first liquid and a second liquid by:
providing the first liquid in a first container, wherein the first liquid comprises at least one lipid;
providing the second liquid in a second container, wherein the second liquid comprises water;
directing the first liquid along a first fluid line into a first inlet of a micromixer and in a flow up to an outlet of the micromixer; and
directing the second liquid along a second fluid line into a second inlet of the micromixer and in a flow adjacent to the first liquid up to the outlet of the micromixer;
wherein the first liquid and the second liquid mix inside the micromixer, so that the liquid with liposomes is discharged at the outlet of the micromixer; further wherein the directing of the first liquid and of the second liquid into the micromixer and up to the outlet of the micromixer is performed by gas pressure from at least one gas source, further wherein the total flow rate of the liquids is adjusted so that it is at least 10 mL/min at the outlet of the micromixer.

20. The liquid according to claim 15, wherein the liquid is used:
in cosmetics; or
as an additive in a food product.

21. The liquid according to claim 15, wherein the liquid is used:
for encapsulating at least one substance in a first micromixer or in a further mixer, after assembling the liposomes or after purifying the liposomes;
for targeting at least one substance;
for releasing at least one substance; or
producing a composite material.
